(19) United States
(12) Patent Application Publication
(10) Pub. No.: US 2025/0049517 A1
(43) Pub. Date: Feb. 13, 2025

(54) ROBOT-SUPPORTED REGISTRATION METHOD AND SURGICAL NAVIGATION SYSTEM

(71) Applicant: B. Braun New Ventures GmbH, Freiburg im Breisgau (DE)

(72) Inventors: Maximilian Dippel, Freiburg (DE); Jean Stawiaski, Kirchzarten (DE)

(21) Appl. No.: 18/723,134

(22) PCT Filed: Dec. 13, 2022
(86) PCT No.: PCT/EP2022/085625

§ 371 Date: Jun. 21, 2024

(30) Foreign Application Priority Data
Dec. 23, 2021 (DE) 10 2021 134 553.7

(51) Int. Cl. A61B 34/30; A61B 34/20; A61B 90/00

(52) U.S. Cl. CPC A61B 34/30; A61B 2034/2051; A61B 2034/2055; A61B 2034/2065; A61B 2090/364

(57) ABSTRACT

A method for automatic registration of a patient for surgical navigation includes detecting a pose of a robot head with a robot camera by a tracking camera of a navigation system and following the robot camera; controlling and moving a robot arm such that the robot camera is directed to a region of interest of the patient, and creating an image; checking whether there is a visual connection between the tracking camera and robot head with the robot camera; detecting, when a visual connection is present, a pose of the robot head via the tracking camera, or detecting, when the visual connection is missing, interrupted or reduced, a pose of the robot head via a robot-kinematics tracking system; and executing a registration of the patient with respect to the navigation system. The registration method can be used with a surgical navigation system and a computer-readable storage medium.

### CROSS-REFERENCE TO RELATED APPLICATIONS

This application is the United States national phase entry of International Application No. PCT/EP2022/085625, filed on Dec. 13, 2022, and claims priority to German Application No. 10 2021 134 553.7, filed on Dec. 23, 2021. The contents of International Application No. PCT/EP2022/085625 and German Application No. 10 2021 134 553.7 are incorporated by reference herein in their entireties.

### FIELD

The present disclosure relates to a registration method/registering method for registering a patient and/or an image of a patient to a navigation system, in particular to 3D image data. In addition, the disclosure relates to a surgical navigation system for a surgical intervention on a patient for registration and for following/tracking as well as to a computer-readable storage medium.

### BACKGROUND

Surgical navigation usually involves the use of special instruments that have special marking systems with markers, such as infrared reference points. These instruments are called indicators or pointers and are specially adapted to mark a position in three-dimensional space with their tip, which is detected by a navigation system. In particular, such navigated pointers are used to scan landmarks on a patient's face in order to perform a registration of the patient and/or of an image of the patient with respect to the navigation system to be used.

Another alternative of a registration is the use of a followed (video) camera in combination with a navigation camera/tracking camera.

However, these registration methods have the disadvantage that they work very poorly in a prone position of the patient, since the patient's face is facing downward. In this case, the surgeons have to kneel down and get under the patient in order to detect the patient's face for registration, which leads to problems with a field of view or an (optical) line of sight between the manually guided pointer or video camera and the tracking camera/following camera/navigation camera. In order to establish visual contact between the tracking camera and the followed pointer or followed camera, surgeons often have to reposition the navigation camera/tracking camera several times during registration so that visual contact is re-established. This delays an intervention procedure and also poses safety risks for both the patient and the medical staff.

As an alternative to registration in the prone position, the patient can be registered in a supine or lateral position and then turned into the prone position for the intervention. However, this method is very risky and greatly increases the complexity of the surgical procedure.

Manual registration is also a question of the individual's skills. Based on different individual experiences and skills, the quality of the registration may therefore vary depending on which person carries out the registration. Manual registration is very time-consuming, in particular if the registration has to be repeated several times due to a lack of experience in order to obtain a sufficient result.

### SUMMARY

The objects of the present disclosure are therefore to avoid or at least reduce the disadvantages of the prior art and, in particular, to provide a registration method, a surgical navigation system and a computer-readable storage medium which provides a higher precision of a registration and a navigation and, in particular, allows a continuous and uninterrupted following and preferably an even better detection of an intervention region. A user, in particular a surgeon, is to receive an even better and safer registration and thus navigation modality. In addition, a partial object is to support a surgical workflow even better and to perform it faster. Another partial object can be seen in increasing the available navigation time during an intervention.

The objects of the present disclosure are solved with respect to a generic registration method, with respect to a generic surgical navigation system, and with respect to a computer-readable storage medium.

A basic idea of the present disclosure can thus be seen in the fact that an optical robot camera provided on a robot arm is used for precise registration of a patient (and/or of an image of a patient), which in particular spatially detects and samples/scans a face of the patient. This optical, robot-guided robot camera is in turn detected and followed in its pose by a tracking system, in particular with and via a navigation camera. The tracking system preferably detects the patient, for example via a patient tracker, and detects the robot camera, for example via a robot camera tracker. The robot camera can be guided by the robot to scan relevant body portions of the patient, in particular the face, in different poses from different viewing directions and can create corresponding images. If the robot camera enters a spatial area in which there is no direct visual contact between the tracking system with the tracking camera and the robot head with the robot camera, in particular with the robot tracker, a robot kinematics-based tracking system (robot-kinematics tracking system) can be used and supplemented for following the pose of the robot camera in order to determine a pose of the robot head or of the robot camera relative to the patient and/or relative to the navigation system, in particular to the tracking camera, even without direct visual contact. Since the robot head has a fixed transformation to the robot camera, conclusions can be drawn from one local coordinate system (KOS) to the other local KOS or a pose of the robot head allows conclusions to be drawn about the pose of the robot camera. In particular, a transformation from a robot camera tracker to the robot camera is known to the navigation system or can be determined.

Such a registration method or navigation method and navigation system offer the advantages of a high efficiency of a registration as well as a significant improvement of a reproducibility or repetition of such a registration, since these are carried out automatically and robot-guided. In particular, this reduces the stress factor for surgeons and assistants and an intervention can be performed even more efficiently and quickly. The robot of the navigation system can preferably also implicitly check the accessibility of a working volume or intervention region during the registration process. In particular, the registration method and the navigation system can therefore be used to check the accessibility of a surgical working area for the robot during the registration process as an additional advantage. In this way, repositioning of the robot, such as a mobile robot with a mobile cart as the robot base, can be avoided during the intervention.

In other words, in particular a registration in the field of robotics and a navigation in surgery, in particular neurosurgery, is disclosed. Here, an automatic and precise registration is performed using a (view) camera (robot camera) attached or mounted to a robot arm, which scans the patient's face in particular for registration of the patient to the navigation system. This (view) camera/robot camera is followed by a combination of using a tracking camera/navigation camera of the navigation system and an (integrated) robot tracking system (robot-kinematics tracking system). The surgical navigation system of the present disclosure thus has two different following modalities available for following the robot camera and can choose the best modality or use a combination for even more precise registration.

The major problem that assistants and surgeons have different skills for performing a registration is eliminated in the present registration method and in the surgical navigation system according to the present disclosure. Likewise, registrations do not have to be performed several times or repeated at a later point in time, for example if the surgeon was not satisfied with the work of the assistant, but in particular a new automatic registration can be performed, for example at regular time intervals or at certain points in time during the intervention (at any time), in order to improve both the quality of a registration and its reproducibility and efficiency. In particular, automatic registration can be carried out at all possible times during the intervention in order to improve quality, reproducibility and efficiency.

Whereas in the prior art, manual registration with a manually guided pointer or a followed video camera requires repeated repositioning of a tracking camera of the navigation system, the present registration method and navigation system provides for the use of a robot with a robot arm and a robot camera connected to the robot arm in order to overcome line-of-sight problems without having to move the external tracking system, in particular the navigation camera/tracking camera. Thus, the use of a robot or robot arm is provided for automatic registration by scanning the patient and automatically performing the comparison. In the event of line-of-sight problems or an interruption of a line of sight or field of view between the tracking system and the followed robot camera, the robot positioning system/robot tracking system/robot-kinematics tracking system is used as an intermediate coordinate reference in order to determine the pose of the robot camera via the robot-kinematics tracking system. An intervention with navigation procedures also becomes even safer, as the net navigation time available to the surgeon is further increased.

In other words, a registration method for an (automatic) registration of a patient for surgical navigation in a surgical intervention is disclosed, comprising the steps of: preferably detecting a pose of the patient, in particular a head of the patient, by a tracking camera, in particular a 3D-tracking camera, of a navigation system; detecting (a pose of) a robot head with a robot camera (of a medical robot), in particular via a robot-camera tracker, by the tracking camera of the navigation system and following the (position and/or orientation, in particular the pose of the) robot camera; controlling and moving the robot arm such that the robot camera in a first pose is directed to a region of interest of the patient, in particular to a face of the patient, and creating an image by the robot camera; checking whether there is a visual connection between the tracking camera and the robot head with the robot camera, in particular with the robot tracker, in the first pose; detecting, when there is a visual connection, a pose of the robot head with the robot camera via the tracking camera, or detecting, when the visual connection is missing or interrupted or reduced, a pose of the robot head with the robot camera via a robot-kinematics tracking system; and executing a registration of the patient and/or of the image (created by the robot camera) with respect to the navigation system, in particular with respect to the tracking camera. In particular, during registration, the image created by the robot camera is converted into coordinates or into a coordinate system of the tracking camera.

One could also say that the registration method or the navigation system for following (of a pose) primarily uses the tracking camera, but switches to a second mode of following when a visual contact deteriorates, i.e. to following based on the robot-kinematics tracking system.

The term “position” means a geometric position in three-dimensional space, which is specified in particular via coordinates of a Cartesian coordinate system. In particular, the position can be specified by the three coordinates X, Y and Z.

The term “orientation” in turn indicates an orientation (such as position) in space. It can also be said that the orientation indicates a direction or rotation in three-dimensional space. In particular, the orientation can be indicated via three angles.

The term “pose” includes both a position and an orientation. In particular, the pose can be specified using six coordinates, three position coordinates X, Y and Z and three angular coordinates for the orientation.

The term 3D defines that the data or structures are spatial, i.e. three-dimensional. The patient's body or at least a part of the body with a spatial extension can be digitally available as image data in a three-dimensional space with a Cartesian coordinate system (X, Y, Z), for example.

In particular, no tracking camera of the navigation system is required if only the robot-kinematics tracking system of the robot is used. In particular, a transformation between the robot base and the patient is already known or can be determined via the robot camera.

According to a further variant, the registration method may further comprise the steps of: controlling and moving the robot arm such that the robot camera in at least a second pose is directed to a further region of interest of the patient, in particular to a further facial region of the patient, and creating a second image by the robot camera; checking whether in the second pose there is a visual connection between the tracking camera and the robot head with the robot camera, in particular with the robot tracker; detecting, when there is a visual connection, the second pose of the robot head with the robot camera via the tracking camera, or detecting, when the visual connection is missing or interrupted or reduced, a pose of the robot head with the robot camera via the robot-kinematics tracking system. In particular, the registration method can run through several poses of the robot camera in succession and can create a corresponding image in each pose. In particular, a continuous movement with continuous followed poses of the robot camera and continuous images, similar to a video recording, can be created.

Preferably, in the step of detecting the pose, when a visual connection is missing or interrupted or reduced, the detection of the pose of the robot head with the robot camera may be performed via the robot-kinematics tracking system, wherein a transformation between a robot base of the robot and the tracking camera is used in order to determine a transformation between the local coordinate system of the tracking camera and the coordinate system of the robot camera and thus its pose via the transformation from the tracking camera to the robot base and the transformation of the robot-kinematics tracking system from the robot base to the robot head with the robot camera.

According to one embodiment, the registration method may further comprise the step of: detecting a relative pose of the robot head with the robot camera to the pose of the head of the patient via the tracking camera in order to provide the robot with an orientation for automatic registration.

In particular, the registration method may further comprise the step of: using a 3D camera as a robot camera and scanning a face of the patient, in particular a head of the patient, from multiple sides in order to obtain an overall image of the face, in particular of the head.

In particular, the registration method may have the step of detecting a pose of the robot base and thus a reference coordinate system of the robot and the robot-kinematics tracking system by the tracking camera. In particular, the navigation system can also detect a pose of the patient, in particular of the patient's head, via the tracking camera, or the registration method may have this step, so that a transformation between the local coordinate system (KOS) of the robot and the local coordinate system (KOS) of the patient is known, and the robot-kinematics tracking system can act independently of the tracking camera.

According to a further embodiment, the step of registering the patient can only be carried out once the images have been created and the robot camera is back in the field of view of the tracking camera.

With regard to a surgical navigation system for a surgical intervention on a patient, the objects of the present disclosure are solved in particular by comprising: a robot with a robot base as a local connection point, a robot arm connected to the robot base and a robot head connected to the robot arm, in particular mounted, with a robot camera, so that a pose (i.e. a position and orientation) of the robot camera relative to the robot base is adjustable; a tracking system with a tracking camera adapted for following (a pose of) preferably a patient, in particular a head of the patient, preferably via a patient tracker, and for following the robot camera (10), in particular via a robot camera tracker; a robot-kinematics tracking system adapted to determine a relative pose of the robot head with the robot camera with respect to its robot base; and a control unit adapted to control the robot and to perform a registration of the patient, wherein the control unit is configured: in a first pose of the robot camera, to create an image by the robot camera, in particular of a face of the patient; when there is a visual connection between the tracking camera and the robot head with the robot camera, in particular with a robot-camera tracker, to detect the pose of the robot camera by the tracking camera (in particular relative to the latter) or, when the visual connection is missing or interrupted or reduced, to detect the pose of the robot camera (in space) via the robot-kinematics tracking system; and to perform a registration of the patient and/or of the image of the robot camera with respect to the navigation system, in particular with respect to the tracking camera. In particular, during registration, the image created by the robot camera has to be transferred to the coordinates or coordinate system (KOS) of the navigation system, in particular of the tracking camera. In particular, a transformation is determined between the coordinate system of the image of the robot camera and the coordinate system of the navigation system, in particular the KOS of the tracking camera.

According to a further embodiment, the surgical navigation system may comprise a robot-guided surgical microscope as a robot and a microscope head of the surgical microscope may function as a robot head with a robot camera. In other words, in particular, the robot camera used for registration may be a surgical microscope or a microscope head of the surgical microscope with an optical system and a sensor for a corresponding image.

Preferably, the surgical navigation system may comprise an infrared-based (optical) tracking system and/or may comprise an electromagnetic (EM) or EM-based tracking system and/or may comprise a machine vision-based tracking system or may be based on machine vision.

With respect to a computer-readable storage medium, the objects are fulfilled by comprising instructions which, when executed by a computer, cause the computer to perform the method steps of the registration method according to the present embodiment.

Any disclosure related to the navigation system of the present disclosure applies equally to the registration method of the present disclosure and vice versa.

### BRIEF DESCRIPTION OF THE DRAWINGS

The present invention is explained in more detail below with reference to the accompanying Figures via preferred configuration examples.

FIG. 1 shows a schematic view of a surgical navigation system according to a preferred embodiment, which uses infrared tracking to detect a transformation between a robot head and a patient head;

FIG. 2 shows a further perspective view of the navigation system from FIG. 1, wherein the camera with depth perception/distance detection is used to scan the patient from all relevant sides;

FIG. 3 shows a further perspective view of the navigation system from FIGS. 1 and 2, wherein the robot base is temporarily used as a reference for line-of-sight problems with the robot tracker and the pose of the robot head is determined via the kinematics;

FIG. 4 shows a further perspective view of the navigation system of FIGS. 1 to 3, wherein a registration is performed as soon as the scan is completed and the robot tracker is visible again; and

FIG. 5 shows a flow diagram of a registration method of a preferred embodiment.

The Figures are schematic in nature and are intended only to aid understanding of the invention. Identical elements are marked with the same reference signs. The features of the various embodiments can be interchanged.

### DETAILED DESCRIPTION

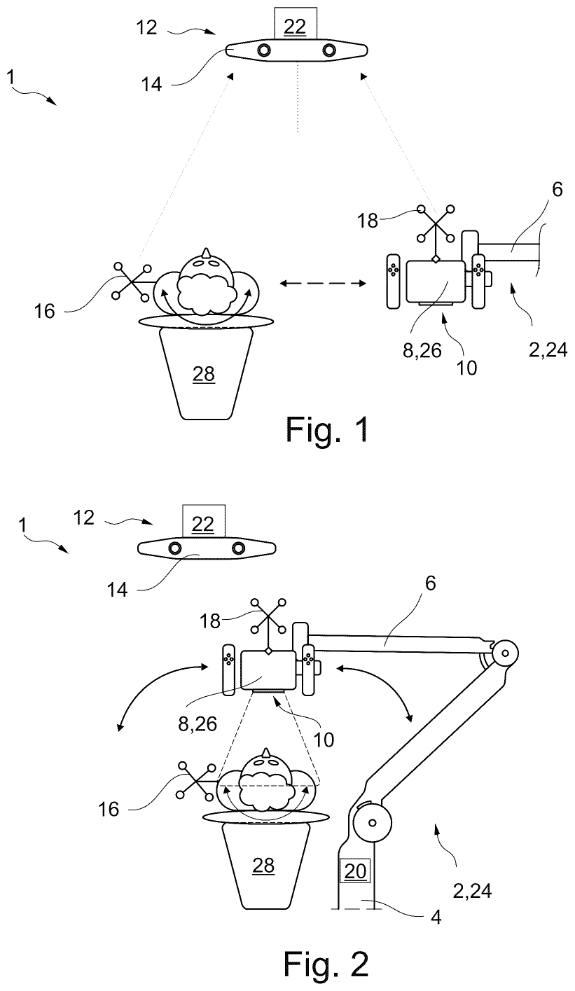

FIGS. 1 to 4 show, for the purpose of explaining the present disclosure and its principles, a schematic perspective view of a surgical navigation system 1 for navigating during a surgical intervention on a patient P according to a preferred embodiment, which can perform automatic registration of a patient.

The navigation system 1 has a robot 2 with a robot base 4 as a local connection point. A multi-jointed robot arm 6 with several robot arm segments is connected to the robot base 4 to enable articulation. In turn, a terminal robot head 8, which can be moved to different positions in different orientations in the area of the patient, is attached to the robot arm 6. The robot head 8 in turn has a robot camera 10 at the front, which can create optical images, in this case three-dimensional images, i.e. 3D images with depth information. The robot arm 6 and the robot head 8 can also be used to set a pose, i.e. a position and orientation of the robot camera 10 in relation to the robot base 4 and thus also in relation to the patient P.

For detecting a position and a pose of objects, the navigation system 1 has an infrared-based tracking system 12, which has a tracking camera 14 at such a position in an operating room that it has a good overview of the intervention region, in particular is arranged in an elevated position. In the present case, the tracking camera 14 is a 3D tracking camera in the form of a stereo camera, so that a pose of an object to be tracked can be detected via two cameras spaced apart from each other and corresponding processing of the images, in particular via triangulation.

In this embodiment, infrared (IR) markers/IR markers or IR trackers are arranged on the objects to be tracked. These IR markers have four rigidly spaced IR spheres for position and orientation detection. Specifically, a patient tracker 16 in the form of an IR tracker is arranged on a patient's head and a robot camera tracker 18 is also arranged on the robot head 8 with the robot camera 10. This enables the (stereo) tracking camera 14 of the navigation system 1 to determine and track the pose of the patient and the pose of the robot camera 10 via a predefined relationship between the tracker and the patient's head or the robot camera.

In addition to the optical, infrared-based tracking system 12, the present navigation system 1 also has a robot-kinematics tracking system 20 of the robot, which is adapted to determine a relative pose of the robot head 8 with the robot camera 10 in relation to its robot base 4. Specifically, sensors are installed in the robot 2 at the joints, which detect a joint position of the robot 2 and provide these settings of the joints in a computer-readable form. A processing unit, here formed by a central control unit 22, then determines the pose of the robot head 8 on the basis of the detected position of the robot and then the relative pose (of the local coordinate system (KOS)) of the robot camera 10 in relation to (the local coordinate system (KOS)) of the robot base 4 via a known transformation from robot head 8 to robot camera 10.

The control unit 22 is further adapted to control the robot 2 and to perform a registration of the patient P, wherein the control unit 22 is configured to control and move the robot camera 10 into a first pose and to create an image by the robot camera 10 in this first pose of a face of the patient P. Thus, a 3D surface image is created by the robot camera 10 in a first pose.

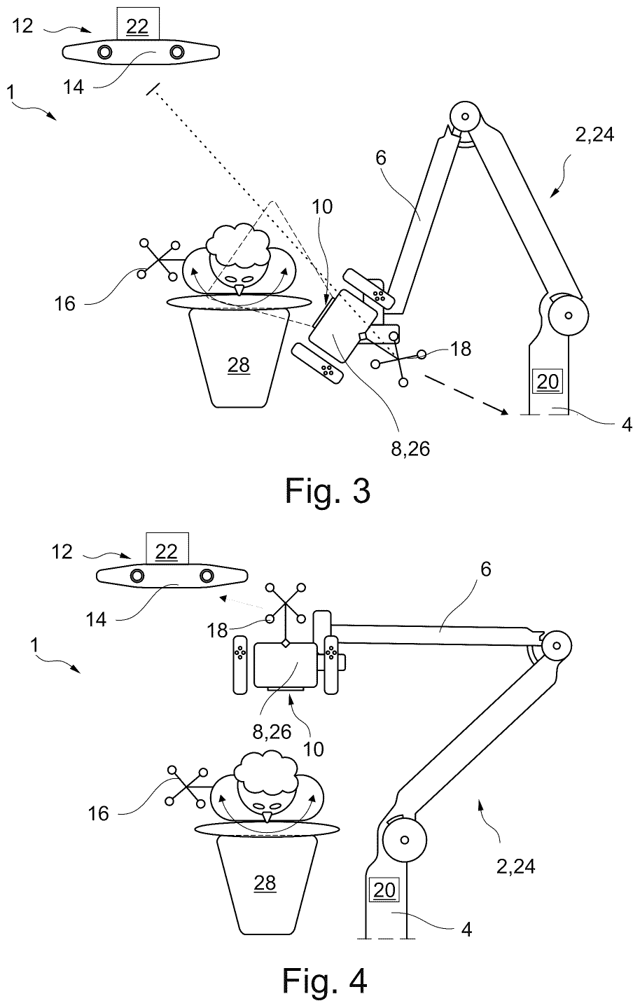

If, as shown in FIGS. 2 and 3, there is a direct visual connection/line of sight between the tracking camera 14 and the robot camera tracker 18, i.e. if the tracking system 12 can locate the robot camera tracker 18 and thus the robot head 8 with the robot camera 10 sufficiently well via the tracking camera 14, the navigation system uses the tracking system 12 with the tracking camera 14 to detect the pose of the robot camera 10 in space. On the one hand, this determines the relative pose of the robot camera 10 in relation to the local coordinate system (KOS) of the tracking system 2 or of the tracking camera 14, and on the other hand, since the pose of the patient P or at least a body portion of the patient P, in this case the head of the patient P, has also been detected in relation to the tracking system 12 or the tracking camera 14, the pose of the robot camera 10 in relation to the patient P can also be determined. In particular, a pose of a 3D surface image of the robot camera 10 relative to the patient can then also be determined and used for registration. This means that if there is a visual connection between the tracking camera 14 and the robot head 8 with the robot camera 10, in this case the robot camera tracker 18, the pose of the robot camera 10 in relation to the navigation system 1, in particular the tracking system, is detected by the tracking camera 14.

If, on the other hand, such a visual connection is missing or is interrupted or severely impaired/reduced, the present navigation system 1 also has a robot-based or kinematics-based tracking system, i.e. the robot-kinematics tracking system 20, in addition to the optical tracking system. This provides a relative pose of the robot camera 10 in relation to the robot base 4. The tracking system 12 can determine a transformation between the local coordinate system of the tracking system 12 or the tracking camera 14 and the local KOS of the robot base 4 via a predetermined transformation between the tracking camera 14 and the robot base 4 or via direct detection of the robot base 4 in visual connection/line of sight, in particular via a robot base tracker (not shown here). This means that in poses of the robot head 8 and the robot camera 10 in which the line of sight between the tracking camera 14 and the robot camera 10 or indirectly via the robot camera tracker 18 is disturbed (see FIG. 3), the robot-kinematics tracking system 20 can be used to automatically register the patient with the navigation system 1 (see FIG. 4).

In the embodiment according to FIGS. 1 to 4, the navigation system 1 has a robot-guided surgical microscope 24 as robot 2. A microscope head 26 of the surgical microscope 24 with the optical system and the image sensor (not shown here, as it is located inside the microscope head 26) serves as robot head 8 with robot camera 10.

In FIG. 1, the navigation system first uses infrared-based tracking to evaluate a spatial, geometric relationship and transformation between the robot head 8 and the patient's head P in order to show the robot the possible working area and to position the robot 2 accordingly.

In FIG. 2, the robot camera 10 (3D image camera or camera with depth perception/depth detection) is then used to scan the patient, in particular the head of the patient P, preferably the face of the patient P, from all relevant sides and to create a 3D surface image of at least the face. Since the robot-camera tracker 18 is always in direct visual contact with the tracking camera 14 in the positions above the patient P, it can also determine the pose of the robot camera 10 relative to the tracking system 12 or the tracking camera 14 (e.g. a lens of the tracking camera 14 as a local KOS of the tracking system).

If, as shown in FIG. 3, the patient is placed or turned into the prone position before or during an intervention, a line-of-sight problem arises because the robot camera 10, when it detects the downward-facing face of the patient P, is positioned below the patient P and the patient P or a surgery table 28 or other medical personnel or even the robot 2 itself blocks a visual connection to the tracking camera 14. In such line-of-sight problems with the robot-camera tracker 18, the robot base 4 is therefore temporarily used as a reference and the pose of the robot camera 10 relative to the patient P is detected via the robot-kinematics tracking system 20. In the present embodiment, the pose of the robot camera 10 is detected via the robot base and further via the tracking camera 14 toward the patient P.

Once all relevant images of patient P's face have been created and the 3D surface scan has been completed, registration is carried out and completed as soon as the robot-camera tracker 18 is back in the field of view of the tracking camera 14.

This registration process may be repeated at various times before or during the intervention in order to achieve particularly accurate registration.

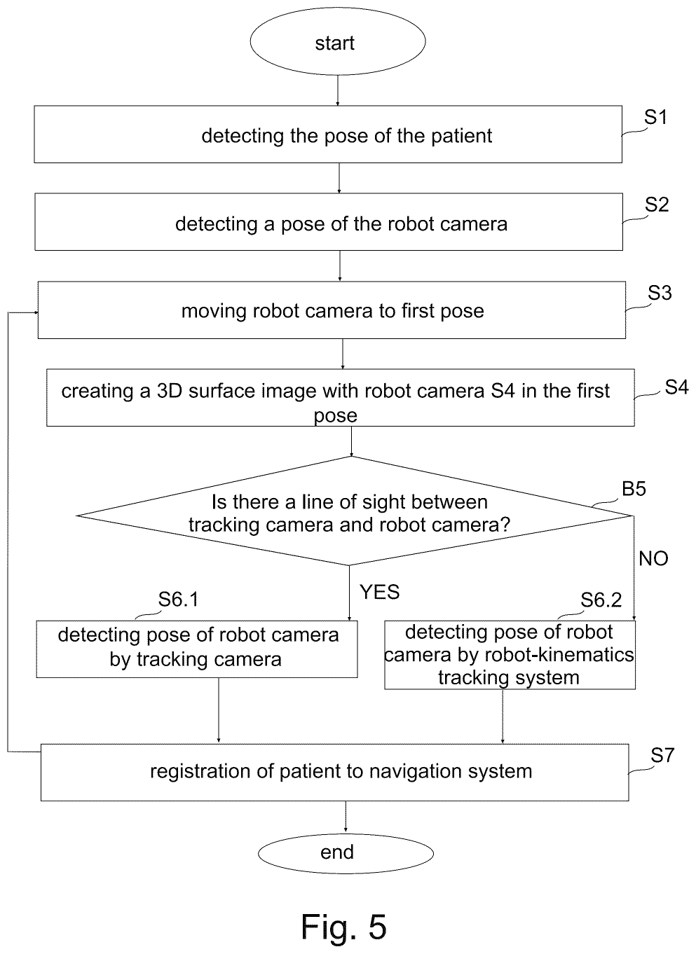

FIG. 5 shows in a flowchart a registration method according to a preferred embodiment, which can be carried out in a surgical navigation system, in particular in a navigation system 1 described above.

The registration method for an automatic registration of a patient P for a surgical navigation of a surgical intervention has as a (here first) step S1 a detecting of a pose of the patient P, in particular of a head of the patient P, by a tracking camera 14, in particular a 3D-tracking camera, of a surgical navigation system 1.

In a subsequent step S2, a pose of a robot head 8 is detected with a robot camera 10 of a medical robot 2, via a robot camera tracker 18, by the tracking camera 14 of the navigation system 1 and following the robot camera 10.

In a step S3, the robot arm is controlled and moved accordingly in such a way that the robot camera 10 is directed in a first pose toward a region of interest of the patient P, in particular toward a face of the patient P.

In a step S4, an image, in this case a 3D surface image, is then created by the 3D-capable robot camera 10.

In a condition B5, the condition is checked as to whether in the first pose there is a visual connection between the tracking camera 14 and the robot head 8 with the robot camera 10, in this case a visual connection to the robot-camera tracker 18.

If the check is positive and there is a visual connection, the registration method proceeds to step 6.1, which detects the pose of the robot head 8 with the robot camera 10 via the tracking camera 14, in this case via the robot-camera tracker 18.

If the visual connection is interrupted or severely impaired so that the pose can no longer be determined with sufficient accuracy via the tracking camera 14, the pose of the robot head 8 is determined in step S6.2 with the robot camera 10 via a robot-kinematics tracking system 20. Here, a pose of the robot camera 10 can either be determined by the robot-kinematics tracking system 20 in relation to the patient P completely without the tracking camera 14, or the transformation can be provided implicitly by integrating the tracking camera 14 and a corresponding calculation of robot camera 10—robot head 8—robot base 4—tracking camera 14—patient P.

Finally, in step S7, when the robot camera 10 is back in the field of view of the tracking camera 14, the patient P is registered with the navigation system 1.

### LIST OF REFERENCE SIGNS

1 surgical navigation system
2 robot
4 robot base
6 robot arm
8 robot head
10 robot camera
12 tracking system
14 tracking camera
16 patient tracker
18 robot-camera tracker
20 robot-kinematics tracking system
22 control unit
24 surgical microscope
26 microscope head
28 surgery table
P patient
S1 step detecting a pose of the patient
S2 step detecting a pose of the robot camera
S3 step moving robot camera in first pose
S4 step creating an image
B5 condition is there a visual connection?
S6.1 step detecting pose robot camera by tracking camera
S6.2 step detecting pose robot camera by robot-kinematics tracking system
S7 step registration of patient

1. A registration method for an automatic registration of a patient for a surgical navigation, the registration method comprising the steps of:
detecting a position and/or orientation of the patient by a tracking camera of a surgical navigation system;
detecting a pose of a robot head with a robot camera of a medical robot by the tracking camera of the surgical navigation system and following the robot camera;
controlling and moving a robot arm such that the robot camera in a first pose is directed to a region of interest of the patient and creating an image by the robot camera;
checking whether there is a visual connection between the tracking camera and the robot head with the robot camera in the first pose;
detecting, when there is a visual connection, a pose of the robot head with the robot camera via the tracking camera, or detecting, when the visual connection is missing or interrupted or reduced, a pose of the robot head with the robot camera via a robot-kinematics tracking system; and
executing a registration of the patient and/or of the image with respect to the surgical navigation system.

2. The registration method according to claim 1, wherein the registration method further comprises the steps of:
controlling and moving the robot arm such that the robot camera in a second pose is directed to a further region of interest of the patient and creating a second image by the robot camera;
checking whether in the second pose there is a visual connection between the tracking camera and the robot head with the robot camera;
detecting, when there is a visual connection, the second pose of the robot head with the robot camera via the tracking camera, or detecting, when the visual connection is missing or interrupted or reduced, a pose of the robot head with the robot camera via the robot-kinematics tracking system.

3. The registration method according to claim 1, wherein, in the step of detecting the pose, when a visual connection is missing or interrupted or reduced, a detection of the pose of the robot head with the robot camera is performed via the robot-kinematics tracking system, wherein a transformation between a robot base of the medical robot and the tracking camera is used in order to determine a transformation between a local coordinate system of the tracking camera and the local coordinate system of the robot camera and thus its pose via the transformation from the tracking camera to the robot base and a transformation of the robot-kinematics tracking system from the robot base to the robot head of the robot camera.

4. The registration method according to claim 1, wherein the registration method further comprises the step of:
detecting a relative pose of the robot head with the robot camera to the pose of a head of the patient via the tracking camera, in order to provide the medical robot with an orientation for automatic registration.

5. The registration method according to claim 1, wherein the registration method further comprises the step of:
using a 3D camera with depth information as a robot camera and scanning a face of the patient from multiple sides in order to obtain an overall 3D surface image of the face.

6. The registration method according to claim 1, wherein the step of registering the patient is performed when images have been created and the robot camera is again in a field of view of the tracking camera.

7. A surgical navigation system for a surgical intervention on a patient, the surgical navigation system comprising:
a robot with a robot base, a robot arm connected to the robot base and a robot head connected to the robot arm with a robot camera, so that a pose of the robot camera relative to the robot base is adjustable;
a tracking system with a tracking camera adapted for following a position and/or orientation of a patient and for following a pose of the robot camera;
a robot-kinematics tracking system adapted to determine a relative pose of the robot head with the robot camera with respect to its robot base; and
a control unit adapted to control the robot and to perform registration of the patient, wherein the control unit is configured:
in a first pose of the robot camera, to create an image by the robot camera to align the robot camera with a face of the patient and to create an image of the face of the patient;
when there is a visual connection between the tracking camera and the robot head with the robot camera to detect the pose of the robot camera by the tracking camera or, when the visual connection is missing or interrupted or reduced, to detect the pose of the robot camera via the robot-kinematics tracking system; and
to perform a registration of the patient and/or of the image with respect to the surgical navigation system based on the image and the pose.

8. The surgical navigation system according to claim 7, wherein the surgical navigation system has a surgical microscope that is robot-guided and a microscope head of the surgical microscope that functions as the robot head with the robot camera.

9. The surgical navigation system according to claim 7, wherein the surgical navigation system comprises an infrared-based optical tracking system.

10. (canceled)

11. The surgical navigation system according to claim 7, wherein the surgical navigation system comprises an EM-based tracking system.

12. The surgical navigation system according to claim 7, wherein the surgical navigation system comprises a machine vision-based tracking system.

13. The surgical navigation system according to claim 7, the surgical navigation system comprises:
an infrared-based optical tracking system; and/or
an EM-based tracking system; and/or
a machine vision-based tracking system.

14. A computer-readable storage medium comprising instructions which, when executed by a computer, cause the computer to perform steps of the registration method according to claim 1.
